(19) United States
(12) Patent Application Publication
(10) Pub. No.: US 2004/0029906 A1
(43) Pub. Date: Feb. 12, 2004

(54) Inhibitors of dna polymerase sigma

(76) Inventors: Michael Christman, Sudbury, MA (US); Sidney M. Hecht, Charlottesville, VA (US); Carrie W. Adams, Royersford, PA (US); Zhenghe Wang, Baltimore, MD (US)

Correspondence Address: UNIVERSITY OF VIRGINIA PATENT FOUNDATION, 1224 WEST MAIN STREET, SUITE 1-110, CHARLOTTESVILLE, VA, 22903, US

(21) Appl. No.: 10/343,563

(22) PCT Filed: Jul. 31, 2001
(86) PCT No.: PCT/US01/23908

(51) Int. Cl. A61K 31/56; A61K 31/4745

(52) U.S. Cl. 514/283; 514/169; 514/559

(57) ABSTRACT

The present invention relates to an improved camptothecin composition for treating a patient having a disease associated with underised cell growth or proliferation, including for example cancer. More particularly, the present invention is directed to a composition comprising camptothecin or a camptothecin-related compound and a DNA polymerase sigma inhibitor.

This application claims priority under 35 U.S.C. §119(e) to provisional patent application No. 60/222,263, filed Jul. 31, 2000, the disclosure of which is incorporated herein by reference in its entirety.

### US GOVERNMENT RIGHTS

This invention was made with United States Government support under Grant Nos. RO1 GM46877 and RO1 CA24363, awarded by the National Institutes of Health. The United States Government has certain rights in the invention.

### FIELD OF THE INVENTION

The present invention is directed to inhibitors of DNA Topoisomerase Related Function Proteins (such as DNA polymerase sigma), compositions comprising such inhibitors, and the use of such compositions as therapeutics for treating neoplastic diseases.

### BACKGROUND OF THE INVENTION

Camptothecin is an alkaloid, which was isolated by Wall et al (J. Am. Chem. Soc. 88, 3888-3890 (1966)) for the first time from the tree Camptoteca acuminata, of the Nyssaceae family. The molecule consists of a pentacyclic structure having a lactone in the ring E, which is essential for cytotoxicity. The drug demonstrated a wide spectrum of antitumor activity, in particular against colon tumors and other solid tumors and leukemias, and the first clinical trials were performed in the early 70's. In addition, camptothecin and derivatives are not affected by MDR1-mediated drug resistance.

Studies conducted in S. cerevisiae (yeast) have indicated that the antitumor activity of camptothecin and its related compounds relates to their ability to inhibit DNA topoisomerase I activity [see Nitiss, J. and Wang, J. C. (1991) Yeast as a genetic system in the dissection of the mechanism of cell-killing by topoisomerase-targeting anticancer drugs. In DNA Topoisomerase in Cancer, ed. P Milan, K. W. Kohn, pp. 77-90. New York: Oxford University Press]. Topoisomerases control the topological state of DNA, and more particularly through the introduction of transient strand breaks in the DNA phosphodiester backbone they effect DNA relaxation.

Based on their mode of strand scission, the topoisomerases can be classified into two groups: type I enzymes mediate the reversible single-strand breakage of DNA, while type II topoisomerases transiently break both strands of the DNA duplex. Through the relaxation of supercoils in DNA structure, the eukaryotic DNA topoisomerases facilitate DNA replication and transcription, and are also implicated in DNA recombination. Mechanistic investigations verified that camptothecin (CPT) produced DNA strand breaks in cultured cells and that CPT binds non-covalently to the topoisomerase I-DNA covalent binary complex.

Although camptothecin initially showed great promise as an anticancer agent, clinical trials in phase I and II, were not completed because of the high toxicity showed by the compound, including hemorrhagic cystitis, gastrointestinal toxicity, such as nausea, vomit, diarrhoea, and myelosuppression, especially leucopenia and thrombocytopenia. However, derivatives of camptothecin have been prepared (see for example U.S. Pat. Nos. 6,242,457, 6,228,855 and 6,218,399 the disclosures of which are incorporated herein) including topotecan, 9-amino-camptothecin, 9-amino-10,11 -methylenedioxy-camptothecan and 10, 11-methylenedioxy-camptothecan, 7-ethyl-10-hydroxy 20(S)-camptothecin, and other 7, 9, 10, 11-substituted compounds. These derivative compounds have shown great promise in phase II clinical trials, and topotecan is currently in phase III clinical trials.

As with many antitumor agents, toxicity at high doses is a major limitation to their use. Thus, physicians are actively seeking other chemotherapy agents to be used in combination with camptothecin and its derivatives to enhance the efficacy of these compounds. Anti-DNA topoisomerase II agents such as adriamycin have not shown the desired synergistic effects thus far. However, 3-aminobenzamide, an inhibitor of poly (ADP-ribose) polymerase (an enzyme that may affect the repair of TOP1/camptothecin-induced breaks) does show promise as a synergistic agent with camptothecin.

The present invention is directed to the use of inhibitors of DNA Topoisomerase Related Function Proteins (such as DNA polymerase sigma) as “response modifiers” to reduce the level of camptothecin (and derivatives thereof) required to kill tumor cells and provide enhanced therapeutic benefit.

Definitions

In describing and claiming the invention, the following terminology will be used in accordance with the definitions set forth below.

As used herein the phrase, “DNA Topoisomerase Related Function Proteins” includes any natural product that interacts with topoisomerase I and is necessary for viability in a cell lacking topoisomerase I activity.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

As used herein, “effective amount” means an amount sufficient to produce a selected effect. For example, an effective amount of a DNA polymerase inhibitor is an amount sufficient to cause a reduction in the number of polynucleotides synthesized by a DNA polymerase in an in vitro synthesis reaction after a predetermined length of time.

As used herein, “DNA polymerase sigma inhibitor” and like terms refers to natural and synthetic compounds that decrease the ability of human or yeast polymerase sigma to synthesize a polynucleotide relative to a reaction run in the absence of the compound. The decrease in synthesis may either be a reduction in the average length of the polynucleotide synthesized or a reduction in the number of polynucleotides synthesized within a predetermined length of time.

As used herein the term “polynucleotide” refers to a chain of at least 15 chemically linked nucleotides.

As used herein, the term “treating” includes preventing, alleviating, or curing a malady, disorder, affliction, disease or injury in a patient.

The term, “parenteral” means not through the alimentary canal but by some other route such as subcutaneous, intramuscular, intraspinal, or intravenous.

As used herein, the term “purified” and like terms relate to the isolation of a molecule or compound in a form that is substantially free of contaminants normally associated with the molecule or compound in a native or natural environment.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein the phrase “potentiate the cytotoxicity of a topoisomerase I inhibitor” and similar language relates to enhancing the cytotoxicitiy and/or enhancing the specificity of the topoisomerase I inhibitor for a particular cell or tissue. For example, potentiating the cytotoxicity of the topoisomerase I inhibitor camptothecin includes enhancing camptothecin's efficacy as an anti-tumor agent.

### SUMMARY OF THE INVENTION

DNA topoisomerae I (TOP1) is the target of the broad spectrum antitumor agent camptothecin. The present invention relates to new and improved compositions that enhance the effectiveness of camptothecin and its derivatives. More particularly the present invention is directed to a composition comprising 20(S)-camptothecin, or a derivative of 20(S)-camptothecin, and an inhibitor of DNA polymerase sigma, and the use of such a composition for treating diseases.

### BRIEF DESCRIPTION OF THE DRAWINGS

FIGS. 1A and 1B demonstrate the hypersensitivity of TRF4-deficient yeast to camptothecin. Spots represent serial 10-fold dilutions of saturated yeast cultures spotted onto Petri plates and incubated at 30° C. for 2 days in the presence of 10 ug/ml of camptothcin (FIG. 1A) or absence of drug (FIG. 1B). As seen in FIG. 1A, yeast cells partially deficient in pol sigma (trf4 single mutants, trf4::HIS3 and mcd1-1) were hypersensitive to camptothecin relative to wild-type strains.

FIGS. 2A and 2B represent Coomassie stained gels of in vitro synthesis reactions that demonstrate the TRF4 gene encodes a novel DNA polymerase. FIG. 2A shows the results using Trf4 to extend a 5′ end-labeled oligo dT primer (16 mer) that was hybridized to a poly dA template in the presence of dTTP and Mg²⁺. (Lane 1 is a control, lanes 2-4 represent decreasing concentration of the Trf4 protein, respectively and lanes 5-7 demonstrate that a mutant Trf4 protein missing the N-terminal 240 amino acids of the 584 amino acid protein, Trf4Δ240, is completely unable to polymerize nucleotides. FIG. 2B demonstrates that the sigma polymerase activity is not effected by the presence (+) or absence (−) of a neutralizing monoclonal antibody to DNA pol I (lanes 4-6) whereas DNA pol I activity is inhibited by the antibody (lanes 1-3; abbreviations: Ab=type of antibody; N indicates if Ab is neutralizing or not; β=polymerase β; σ=Trf4 protein).

FIG. 3 represents a Coomassie stained gel of in vitro synthesis reactions using pol sigma-I (TRF4 gene product) and conducted in the presence of 9 different polymerase β inhibitors. Lanes 1-4 represent control; betulinic acid; (24E)-3beta-hydroxy-7,24-euphandien-26-oic acid; ursolic acid, respectively. The remaining lanes represent other polymerase β inhibitors that failed to inhibit pol sigma-1.

### DETAILED DESCRIPTION OF THE INVENTION

DNA topoisomerae I (TOP1) is the target of the broad spectrum antitumor agent camptothecin. In an effort to design therapies that would enhance the effectiveness of camptothecin and its derivatives, applicants have been studying the biological function of TOP1 in the yeast S. cerevisiae. These functions have been difficult to elucidate because cells deficient in TOP1 appear normal by a variety of criteria. Thus it was anticipated that TOP1 accomplishes its essential functions in the cell together with other, as yet unidentified, gene products. Several novel gene products that function in concert with TOP1 have been identified. These genes are called TRF for “DNA Topoisomerase Related Function.”

Genetic evidence has demonstrated that the simultaneous absence of TOP1 and any of the TRF genes in S. cerevisiae renders the yeast non-viable. Further, even in the presence of TOP1, the absence of a TRF gene causes the yeast to be hypersensitive to camptothecin analogues. The implication of these findings is that an inhibitor of TRF gene products should potentiate the cytotoxic action of topoisomerase I inhibitors.

Accordingly, one aspect of the present invention is directed the use of inhibitors of TRF gene products in conjunction with known topoisomerase inhibitors to enhance the effectiveness of the topoisomerase inhibitors in limiting cell proliferation and/or growth. In accordance with one embodiment an inhibitor of a TRF gene product is used to sensitize tumor cells to topoisimerase-targeting antitumor drugs such as camptothecin.

In accordance with one embodiment of the invention a composition is provided that comprises a TOP1 inhibitor and an inhibitor of a TRF gene product. Preferably the TOP1 inhibitor is camptothecin or a camptothecin analog or derivative, including for example, hycamtin, camptostar, topotecan, 9-amino-camptothecin, 9-amino-10,11 -methylenedioxy-camptothecan, 10,11- methylenedioxy-camptothecan, 7-ethyl-10-hydroxy 20(S)-camptothecin, and other 7, 9, 10, 11-substituted compounds. This composition can be administered to a patient to treat diseases that are associated with uncontrolled or undesired growth, as seen for example in cancer and restenosis.

One TRG gene product of particular interest is the gene, TRF4. TRF4 encodes a novel DNA polymerase that has been designated DNA polymerase sigma (pol sigma). Pol sigma is present in all eukaryotic cells, and the corresponding yeast genes and human genes have been cloned and sequenced (see Walowsky et al., (1999) Journal of Biological Chemistry, 274, 7302-7308, the disclosure of which is incorporated herein). Human Pol sigma-1 is located on chromosome 5 and a cDNA of 3766 nucleotides has been isolated. Human Pol sigma-2 is located on chromosome 16 and a cDNA of 1375 nucleotides has been isolated. The sequences of these two genes are deposited with Genbank under accession numbers AF089896 for human Pol sigma-1 and AF089897 for human Pol sigma-2. Polymerase σ has limited sequence homology to the DNA polymerase β superfamily, but is believed to have an overall structure that is reasonably similar to polymerase β (Aravind, and Koonin (1999) Nucleic Acids Res. 27, 1609-1618).

The cloned yeast polymerase C genes have been used to purified yeast polymerase σ by expressing the protein as a fusion protein with a peptide tag containing six histidines. The expressed protein is then isolated on a Ni-NTA affinity column. Fractions containing TRF4 protein extended an oligo dT₁₆ primer in a distributive fashion, a property characteristic of DNA polymerase β.

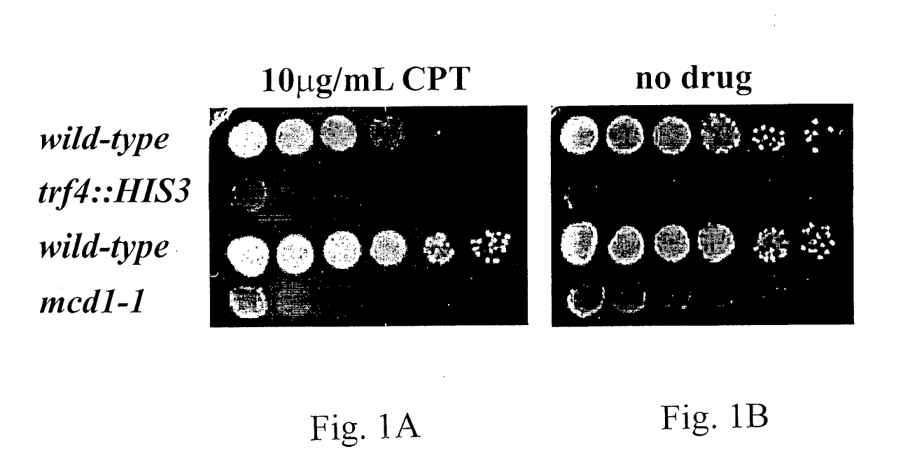

The presence or absence of pol sigma (TRF4 gene product) profoundly affects the cell's sensitivity to the antitumor agent camptothecin. In particular, as shown in FIG. 1, yeast cells lacking TRF4 are 10,000 fold more sensitive to the anti-TOP1 agent. It is anticipated that this will also be true in human cells, and thus inhibitors of human pol sigma should sensitize tumor cells to camptothecin-like compounds.

In accordance with one embodiment a method is provided for potentiating the cytotoxic action of topoisomerase I inhibitors. The method comprises the step of contacting cells with a topoisomerase I inhibitor and a polymerase sigma inhibitor. In one embodiment, the method comprises the steps of contacting cells, in vitro or in vivo, with a composition comprising a pol sigma inhibitor followed contacting the cells with a composition comprising a topoisomerase I inhibitor (or vice versa). Alternatively, the cells are contacted with a single composition comprising a polymerase sigma inhibitor and a topoisomerase I inhibitor. In one embodiment the cytotoxic action of topoisomerase I inhibitors is potentiated with a polymerase sigma inhibitor as part of a therapy for treating neoplastic disease.

The improved camptothecin compositions of the present invention may include any of the know camptothecin derivatives including, but are not limited to, 9-nitro-20(S)-camptothecin, 9-amino-20(S)-camptothecin, 9-methyl-camptothecin, 9chlorocamptothecin, 9-flouro-camptothecin, 7-ethyl camptothecin, 10-methylcamptothecin, 10-chloro-camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, and 10,11-ethylenedioxy camptothecin, and 7-(4-methylpiperazinomethylene)-10,11-methylenedioxy camptothecin. Prodrugs of camptothecin can also be used in this invention and include, but are not limited to, esterified camptothecin derivatives as described in U.S. Pat. No. 5,731,316 (the disclosure of which is incorporated herein), such as camptothecin 20-O-propionate, camptothecin 20-O-butyrate, camptothecin 20-O-valerate, camptothecin 20-O-heptanoate, camptothecin 20-O-nonanoate, camptothecin 20-O-crotonate, camptothecin 20-O-2′,3′-epoxy-butyrate, nitrocamptothecin 20-O-acetate, nitrocamptothecin 20-O-propionate, and nitrocamptothecin 20-O-butyrate.

In particular, when substituted caniptothecins are used, a large range of substitutions may be made to the camptothecin scaffold, while still retaining activity. In a preferred embodiment, the caniptothecin scaffold is substituted at the 7, 9, 10, 11, and/or 12 positions including 9-nitrocamptothecin, 9-aminocamptothecin, 10,11-methylendioxy20(S)-camptothecin, topotecan, irinotecan, 7-ethyl-10-hydroxy camptothecin, or another substituted camptothecin that is substituted at least on one of the 7, 9, 10, 11, or 12 positions.

Native, unsubstituted, camptothecin can be obtained by purification of the natural extract, or it may be obtained from the Stehlin Foundation for Cancer Research (Houston, Tex.). Substituted camptothecins can be obtained using methods known in the literature, or can be obtained from commercial suppliers. For example, 9-nitrocamptothecin may be obtained from SuperGen, Inc. (San Ramon, Calif.), and 9-aminocamptothecin may be obtained from Idec Pharmaceuticals (San Diego, Calif.). Camptothecin and various of its analogs may also be obtained from standard fine chemical supply houses, such as Sigma Chemicals.

In accordance with one embodiment one or more camptothecin or camptothecin derivatives are combined with a polymerase sigma inhibitor to enhance the efficacy of the camptothecin compound as an anti-tumor agent. Suitable compounds for use as pol sigma inhibitors include those compounds that have already demonstrated activity as polymerase β inhibitors. A simple screening assay can be conducted to confirm whether or not such compounds have activity against pol sigma. More particularly the following compounds can be used in accordance with the present invention as pol sigma inhibitors:

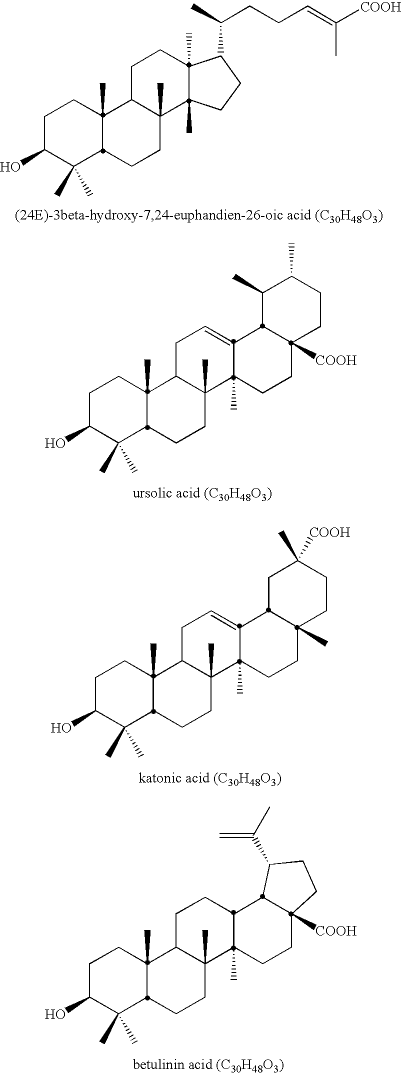

The DNA polymerase sigma inhibitor used in the present invention preferably has an IC₅₀ (inhibitory concentration of drug required to achieve a 50% reduction in polymerase activity) in the micromolar range and more preferably in the nanomolar range or lower. In accordance with one embodiment, the pol sigma inhibitor is selected from the group consisting of (24E)-3beta-hydroxy-7,24-euphandien-26-oic acid, ursolic acid, katonic acid and betulinin acid, and more preferably the pol sigma inhibitor is (24E)-3beta-hydroxy-7,24-euphandien-26-oic acid. This compound has been shown to be a nanomolar inhibitor of yeast pol sigma in vitro. Furthermore, both (24E)-3beta-hydroxy-7,24-euphandien-26-oic acid and ursolic acid have been demonstrated to have inhibitory activity against human pol sigma.

Additional inhibitors of polymerase sigma activity will be identified using two specific assays that are also part of the present invention. In one embodiment the method for isolating an inhibitor of pol sigma comprises the steps of conducting an in vitro DNA synthesis reaction in the presence and absence of a potential inhibitor compound, measuring the amount of DNA synthesized in the two reactions and comparing the amount of DNA synthesized in two separate reactions. In another embodiment, the method relies on a cell based assay comprising the steps of culturing a yeast strain partially deficient in pol sigma (trf4 single mutants, for example) and separately culturing a yeast strain that expresses both isoforms of pol sigma. Both stains are then contacted with the potential inhibitor, and the cell growth of the two strains is then compared.

In accordance with the in vitro assay for identifying inhibitors of polymerase sigma activity, the ability of DNA polymerase sigma to incorporate nucleotides into TCA precipitable material will be measured in the presence and absence of potential inhibitors. In one preferred embodiment fluorescently labeled nucleotides will be used and the amount of fluorescence detected in the a TCA or ethanol precipitation will be indicative of the activity of the polymerase (i.e. only DNA chains longer than about 15 nucleotides will precipitate under appropriate conditions whereas free, unincorporated nucleotides will remain in the supernatant). Such a system can be automated.

In an alternative embodiment, the system used to identify pol sigma inhibitors uses a cell based assay. Yeast cells that are partially deficient in pol sigma (trf4 single mutants) are completely dependent on the second pol sigma gene, TRF5, for viability (Castaño et al., 1996B; Castaño et al., 1996A). Thus, compounds that inhibit pol sigma function when the cells express only one isoform will be preferentially killed over strains that express two isoforms. In one embodiment, compounds that kill (or inhibit the growth of) a yeast trf4 trf5 double mutant that expresses human TRF4-1, but do not kill a yeast strain mutant in trf4 but not TRF5 will be identified as human TRF4-1 inhibitors. This assay ensures that the identified compounds work by acting to inhibit TRF function since its lethal consequences are reversed if TRF5 is present.

Accordingly, one method for identifying pol sigma inhibitors comprises the steps of culturing parallel cultures of yeast expressing either: 1) a single human pol sigma-1 (and deficient in the native yeast pol sigma); or 2) human pol sigma-1 and a second pol sigma gene. The yeast cells will then be contacted with potential polymerase inhibitors and the cultures will be assayed in a 96-well format for optical density following a predetermined growth period. Compounds that preferentially inhibit the growth of yeast expressing only the one human pol sigma gene, and that fail to inhibit strains bearing two copies of pol sigma will be taken as positives.

In one embodiment a composition is provided comprising a topoisomerase I inhibitor selected from the group consisting of camptothecin, hycamtin, camptostar, topotecan, 9-amino-camptothecin, 9-amino-10,11-methylenedioxy-camptothecan and 10,11- methylenedioxy-camptothecan, 7-ethyl-10-hydroxy 20(S)-camptothecin, and an inhibitor of DNA polymerase sigma selected from the group consisting of (24E)-3beta-hydroxy-7,24-euphandien-26-oic acid and ursolic acid, and a pharmaceutically acceptable carrier.

Compositions comprising a pol sigma inhibitor can be used in accordance with one embodiment in a method for treating a patient having a disease associated with undesired cell growth or proliferation. The method comprises the steps of delivering to the patient a therapeutically effective amount of a polymerase sigma inhibitor in combination with a therapeutically effective amount of a topoisomerase I inhibitor, such that the efficacy of the therapy is enhanced through the combined effects of the topoisomerase I inhibitor and the polymerase sigma inhibitor. In one embodiment, the topoisomerase inhibitor is selected from the group consisting of camptothecin or a camptothecin derivative and the polymerase sigma inhibitor is selected from the group consisting of (24E)-3beta-hydroxy-7,24-euphandien-26-oic acid, ursolic acid, betulinic acid and other known polymerase β inhibitors that effectively inhibit pol sigma. This method can be used to treat a variety of diseases, including neoplastic diseases such as acute myelogenous leukemia, cholangiocarcinoma, chronic myelogenous leukemia, lymphoma, melanoma, multiple myeloma, osteosarcoma, gastric sarcoma, glioma, bladder, breast, cervical, colorectal, lung, ovarian, pancreatic, prostrate, stomach cancer and various other types of cancers such as primary tumors and tumor metastasis.

In addition to treating cancer, the compositions of the present invention can be used to treat other diseases characterized by rapid, uncontrolled or excessive/inappropriate cell growth. Such undesirable cellular growth or proliferation is associated with diseases that include restenosis, benign tumors, abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Specific types of restenotic lesions that can be treated using the present invention include coronary, carotid, and cerebral lesions (see U.S. Pat. No 6,191,119, the disclosure of which is incorporated herein). Specific types of benign tumors that can be treated using the present invention include hemangiomas, acoustic neuromas, neurofibroma, trachomas and pyogenic granulomas. Specific types of cancers that can be treated using this invention include acute myelogenous leukemia, bladder, breast, cervical, cholangiocarcinoma, chronic myelogenous leukemia, colorectal, gastric sarcoma, glioma, leukemia, lung, lymphoma, melanoma, multiple myeloma, osteosarcoma, ovarian, pancreatic, prostrate, stomach, or tumors at localized sites including inoperable tumors or in tumors where localized treatment of tumors would be beneficial, and solid tumors. Treatment of cell proliferation due to insults to body tissue during surgery may be possible for a variety of surgical procedures, including joint surgery, bowel surgery, and cheloid scarring. Diseases that produce fibrotic tissue include emphysema. Repetitive motion disorders that may be treated using the present invention include carpal tunnel syndrome. An example of cell proliferative disorders that may be treated using the invention is a bone tumor.

Abnormal angiogenesis that may be may be treated using this invention includes abnormal angiogenesis that accompanies rheumatoid arthritis, psoriasis, diabetic retinopaphy, and other ocular angiogenic diseases such as retinopathy of prematurity (retrolental fibroplastic), macular degeneration, corneal graft rejection, neuroscular glaucoma and Oster Webber syndrome.

The method of treating diseases characterized by rapid, uncontrolled or excessive/inappropriate cell growth comprises the step of contacting cells with a composition comprising a polymerase sigma inhibitor. More particularly the target cells are contacted with a polymerase sigma inhibitor and a topoisomerase 1 inhibitor. In one embodiment the method comprises delivering to a patient a therapeutically effective amount of camptothecin or camptothecin derivative in combination with an effective amount of an inhibitor of pol sigma. More particularly, the method comprises the steps of administering a topoisomerase inhibitor selected from the group consisting of camptothecin, topotecan, camptothecin-11, 9-amino-camptothecin, 9-amino- 10,11 -methylenedioxy-camptothecan and 10,11-methylenedioxy-camptothecan, 7-ethyl-10-hydroxy 20(S)-camptothecin and administering a polymerase sigma inhibitor selected from the group consisting of (24E)-3beta-hydroxy-7,24-euphandien-26-oic acid, ursolic acid. In one preferred embodiment the topoisomerase inhibitor and polymerase sigma inhibitor are combined and administered in a single dosage formulation.

In one embodiment the disease to be treated using the present compositions is cancer. More particularly the compositions of the present invention are used to treat acute myelogenous leukemia, cholangiocarcinoma, chronic myelogenous leukemia, lymphoma, melanoma, multiple myeloma, osteosarcoma, gastric sarcoma, glioma, bladder, breast, cervical, colorectal, lung, ovarian, pancreatic, prostrate, or stomach cancer. Alternatively, the method can also be used to treat non-cancerous diseases that are characterized by excessive/inappropriate cell growth, including the endothelial cell growth associated with restenosis.

The compounds and compositions of the present invention can be administered by a variety of routes, and may be administered or coadministered in any conventional dosage form. Coadministration in the context of this invention is defined to mean the administration of more than one therapeutic (i.e. administration of topoisomerase inhibitor and polymerase sigma inhibitor) in the course of a coordinated treatment to achieve an improved clinical outcome. Such coadministration may also be coextensive, that is, occurring during overlapping periods of time or being administered simultaneously.

More particularly, the topoisomerase inhibitor and polymerase sigma inhibitor of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. Intravenous administration is one preferred method of administering the composition of the present invention. The compounds and/or compositions according to the invention may also be administered or coadministered in slow release dosage forms.

One therapeutic route of administration or coadministration is local delivery. Local delivery of effective amounts of the present composition can be delivered by a variety of techniques and structures that administer the compositions of the present invention at or near a desired site. Examples of local delivery techniques and structures are not intended to be limiting but rather as illustrative of the techniques and structures available. Examples include local delivery catheters, site specific carriers, implants (including slow release formulations), direct injection, or direct applications.

In accordance with one embodiment a kit is provided for treating cancer and other diseases associated with inappropriate cell proliferation and growth. The kit comprises a topoisomerase inhibitor and a polymerase sigma inhibitor. In one embodiment the topoisomerase inhibitor is camptothecin or a camptothecin derivative, and more particularly the camptothecin derivative is selected from the group consisting of camptothecin, topotecan, 9-amino-camptothecin, 9-amino-10,11-methylenedioxy-camptothecan and 10,11-methylenedioxy-camptothecan, 7-ethyl-10-hydroxy 20(S)-camptothecin. The polymerase sigma inhibitor in one embodiment is selected from the group consisting of (24E)-3beta-hydroxy-7,24-euphandien-26-oic acid, ursolic acid, katonic acid, betulinic acid and other known polymerase β inhibitors that effectively inhibit pol sigma. In one embodiment the polymerase sigma inhibitor is (24E)-3beta-hydroxy-7,24-euphandien-26-oic acid or ursolic acid. The inhibitors of the present invention can be packaged in a variety of containers, e.g., vials, tubes, microtiter well plates, bottles, and the like. Other reagents can be included in separate containers and provided with the kit; e.g., positive control samples, negative control samples, buffers, solvents, cell culture media, etc.

### EXAMPLE 1

Identification of a novel DNA polymerase. TRF4

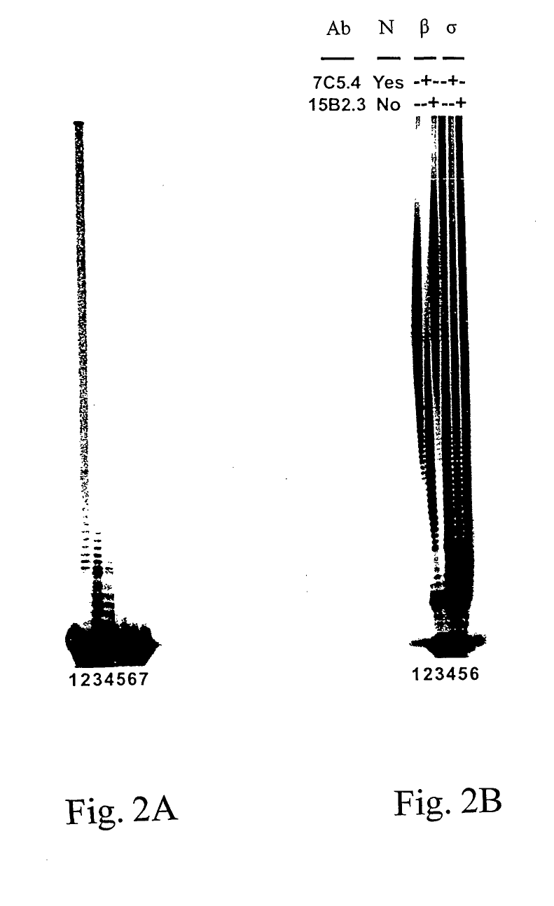

To test the significance of the limited sequence homology between TRF4 and the β-polymerase superfamily, Trf4 fused to a six histidine tag was purified from Escherichia coli to apparent homogeneity and recombinant protein was assayed for DNA polymerase activity. First, Trf4 was examined for the ability to extend a 5′ end-labeled oligo dT primer (16 mer) that was hybridized to a poly dA template (average size 282 nucleotides) in the presence of dTTP and Mg²⁺(FIG. 2A). Trf4 is able to extend the primer in a distributive manner (extension of a single nucleotide followed by dissociation from primer/template), which is characteristic of β-DNA polymerases. In contrast, a mutant Trf4 protein missing the N-terminal 240 amino acids of the 584 amino acid protein, Trf4Δ240, is completely unable to polymerize nucleotides (lanes 5-7 of FIG. 2A).

Fractions eluted from the final mono Q anion exchange column demonstrate cofractionation of Trf4 protein and DNA polymerase activity. The DNA polymerase activity observed is dependent on template, primer and Mg²⁺. To ensure that the activity observed was not due to E. coli pol I contamination (the major polymerase activity in E. coli extracts, a neutralizing monoclonal antibody to DNA pol I was used in the polynierase reactions (Ruscitti et al., 1992, J Biol Chem 267, 16806-11). FIG. 2B shows that incubation of the neutralizing antibody with DNA pol I inhibits its ability to extend the oligo dT primer, whereas a different monoclonal antibody to DNA pol I that does not neutralize the activity has no effect on DNA pol I activity (Ruscitti et al., 1992, J Biol Chem 267, 16806-11). In contrast, Trf4 activity is unaffected by either monoclonal antibody (lanes 4-6 of FIG. 2B). In addition, the size range of the Trf4 products is consistently observed to be greater than for DNA pol I.

Identification of Inhibitors of the novel DNA polymerase sigma

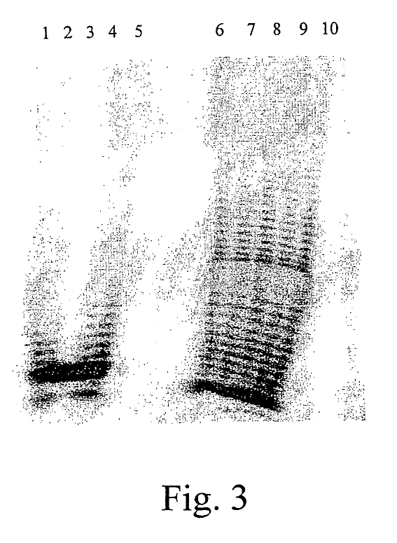

Due to the predicted structural similarity between pol sigma and pol β (Aravind and Koonin, 1999, Nucleic Acids Research 27: 1609-1618) several known β polmerase inhibitors were tested to determine if they would inhibit pol sigma in vitro. The assay system employed for testing whether the known β polmerase inhibitors would inhibit polymerase σ utilized a 5′-³²P end labeled dT₁₆ primer that had been annealed to a poly dA template (average length 282 nucleotides). The template primer was incubated in the presence of dTTP, Mg²⁺ and 10 μM test compound for 5 min. Nine known β polmerase inhibitors were tested for polymerase σ inhibitory activity, and as shown in FIG. 3 the specificity of the inhibition is striking. Lane 1 of FIG. 3 is a control with only the DMSO solvent added to the poly dA/ oligo dT assay, the remaining lanes represent reactions run in the presence of one of the β polmerase inhibitors. As shown in FIG. 3, the inhibitor in lane 3, (24E)-3β-hydroxy-7,24-euphandien-26-oic acid, was the most potent of the species tested, with betulinic acid (lane 2) and ursolic acid (lane 4) also showing activity. In particular, compound (24E)-3beta-hydroxy-7,24-euphandien-26-oic acid (see structure above) is estimated to be a nanomolar level inhibitor of yeast pol sigma polymerase activity.

On the basis of additional experiments, (24E)-3β-hydroxy-7,24-euphandien-26-oic acid was determined to have an IC₅₀ of approximately 500 nM as an inhibitor of yeast polymerase σ, whereas betulinic acid and ursolic acid compounds were found to inhibit yeast polymerase σ at micromolar concentrations. The lack of activity of 6 of the 9 polymerase β inhibitors as inhibitors of polymerase σ indicated that it is possible to obtain selective inhibition of a single polymerase in spite of the putative structural similarity between polymerases β and σ. Further, even for the three compounds that are inhibitory toward both enzymes, the rank order of potencies is quite different. For rat polymerase β, the rank order of potencies was found to be ursolic acid >betulinic acid >> 3β-hydroxy-7,24-euphandien-26-oic acid. Also of interest is that the best pol sigma inhibitor, (24E)-3beta-hydroxy-7,24-euphandien-26-oic acid, has a structure quite different from the other compounds (FIG. 3). It is also known that (24E)-3beta-hydroxy-7,24-euphandien-26-oic acid is active, albeit only at micromolar concentrations as an inhibitor of human pol sigma.

### EXAMPLE 2

Test for human pol sigma inhibitors to potentiate cytotoxicity of camptothecin

To test the hypothesis that an inhibitor of polymerase σ could potentiate the cytotoxicity of topoisomerase I inhibitors such as camptothecin, cells were contacted iin vitro with camptothecin alone or in combination with ursolic acid. Ursolic acid was utilized because it inhibits yeast polymerase σ and it is commercially available.

A mouse (P388D₁) cell line was grown as a suspension culture and treated singly with camptothecin or ursolic acid to determine the greatest concentration of each at which only minimal killing of P388D₁ cells was observed after a 6 hr incubation. Viable cells were scored by the use of a hemacytometer plate after straining with Trypan Blue. As shown in Table 1, minimal toxicity was observed in the presence of 1 μM camptothecin alone or 0.5 μM ursolic acid alone. However, when the compounds were tested together at the same concentrations, the number of viable cells observed was diminished.

TABLE 1
Effect of Camptothecin and Ursolic Acid on the Growth of P388D₁ Cells

| | | % viability |
|---|---|---|
| Compound | Experiment 1 | Experiment 2 |
| -(DMSO control) | 100 | 96 |
| 1 μM camptothecin | 84 | 77 |
| 0.5 μM ursolic acid | 88 | 77 |
| 1 μM CPT + 0.5 μM ursolic acid | 75 | 63 |

### EXAMPLE 3

In vitro screen for agents that inhibit pol sigma function

The polymerase sigma inhibitor compound, (24E)-3beta-hydroxy-7,24-euphandien-26-oic acid, can be used as a tool to help identify other inhibitors of yeast pol sigma in vitro. The lC₅₀ (inhibitory concentration of drug required to achieve a 50% reduction in polymerase activity) of this molecule for yeast pol sigma is in the 500 nanomolar range. At 10 uM concentration no activity whatsoever is observed. Evidence suggests that this compound also work to inhibit human pol sigma-1.

Several approaches will be used to identify additional polymerase sigma inhibitors including: i) identifying inhibitors of human pol sigma gene products based on both high throughput screening of a proprietary chemical extract library together with NCI compound libraries; and ii) combinatorial chemical modification of existing, and further testing of known, β-polymerase inhibitors.

One enormous advantage of secondary screening of potential inhibitors in yeast cell assays is that the potential exists to prove that the pol sigma locus is the sole site of action of a drug, as has been done for topo I and camptothecin. Thus, yeast cells expressing human pol sigma should not be responsive to human pol sigma inhibitors when the cells are carrying a mutant allele of the human enzyme that is not responsive to drug. It should be possible to identify such an allele as 33 surface-targeted mutations in pol sigma have been isolated and analyzed.

High throughput screening for pol sigma inhibitors will also be initiated early in this project for the following reasons: i) the replication fork is a proven target for antitumor agents; ii) because fork components need not be altered in expression in tumors to work in this capacity; iii) because small molecule inhibitors of DNA polymerases have been identified previously; and iv) a nanomolar level inhibitor of yeast pol sigma was already found, suggesting that highly specific inhibitors of this class of enzyme can be identified.

Assays for inhibition of DNA polymerase sigma

A $200,000 robotic, 96-well plate-compatible device for high throughput drug screening is housed in a dedicated lab room at the University of Virginia medical center and will be used for high throughput screening for inhibitors.

It has the ability to read fluorescence and UV in several Z-planes (up and down) and to perform ethanol or TCA precipitations as well as centrifugation in the 96-well format. Assays of two general types that are amenable to screening will be utilized. These are assays for inhibition of DNA polymerase sigma in vitro and assays for pol sigma-specific killing of yeast cell cultures.

Inhibitors of DNA polymerase sigma activity in vitro

In accordance with the in vitro assay, fluorescently labeled nucleotides will be incorporated into TCA precipitable material, an activity possessed by many DNA polymerases. Several different nucleotide-derived fluorophores will be examined. In a control well containing no inhibitory molecules, it is anticipate that the activity of DNA pol sigma will produce a positive signal for TCA (or ethanol) precipitable fluorescence. Only DNA chains longer than about 15 nucleotides will precipitate under appropriate conditions whereas free, unincorporated nucleotides will remain in the supernatant. The precipitated reactions will be centrifuged to facilitate pelleting of DNA chains and then the Z-plane ability of the fluorescence reader will allow for the determination of the extent of the reaction in a highly quantitative manner. It is of course paramount to identify conditions in which the reaction is proportional to time and amount of enzyme so that inhibition assays are robust.

Once the parameters have been optimized, screening will begin using the previously mentioned robotic delivery system that should allow for several thousand assays to be performed per week. A positive hit rate of roughly 1 in 1,000 (0.1%) is anticipated for compounds judged to be “positive” initially. The highly quantitative nature of these assays should make this possible.

Cell Free Assay System

The assay system that has been employed to date employs a dT₁₆ primer annealed to a poly dA template (average length 282 nucleotides). Primer elongation as a consequence of the addition of T residues (in a distributive fashion) has been monitored by polyacrylamide gel electrophoresis. For purposes of screening for inhibitors a variant of the DNA polymerase β this system will be used as follows:

To a 50 μL of 62.5 mM 2-amino-2-methyl-1,3-propanediol buffer, pH 8.6, containing 10 mM MgCl₂, 1 mM DTT, 100 μg/mL BSA, 6.25 μM dNTPs including 0.04 Ci/mmol [³H]dTTP, and 0.25 mg/mL activated calf thymus DNA was added 6 μL of a solution containing each test compound and 4 μL of recombinant rat liver DNA polymerase β preparation (6.9 units, 4.8×10⁴ units/mg). After incubation at 37° C. for 60 min, the radiolabeled DNA product was collected on DEAE-cellulose paper (DE-81), dried, and rinsed successively with 0.4 M K₂HPO₄, pH 9.4, and 95% ethanol for radioactivity determination.

“Activated” calf thymus DNA is prepared by treatment of commercially available DNA preparation with a DNase preparation. It is believe that this assay system constitutes a better model for events in an intact cell than the use of a dTₙ/polydA primer template system, and that it will be a simple matter to develop this system for medium throughput assays as was already done by applicants for polymerase β.

Both the human and yeast DNA polymerase a has been cloned and human polymerase σ will be expressed in an E. coli strain harboring human Trf4-1p/Polσ-1 under the control of a T7 promotor. This strain also has T7 RNA polymerase under the control of a lac promotor (i.e. inducible by IPTG). The elaborated human polymerase σ is a fusion protein containing a hexahistidine motif to facilitate purification by Ni-NTA chromatography. Screening will be done using the human enzyme, however, in order to facilitate correlation of the results with those obtained to date, the yeast enzyme will also be isolated. This can be prepared from an existing E. coli strain harboring yeast Trf4p/Polσ-1, a strain that has already been used to prepare >100 μg of yeast DNA polymerase σ.

Screening of Extracts and Synthetic Compounds

Previously, 3,500 natural products extracts have been screened to identify inhibitors of DNA polymerase β. 26 polymerase β inhibitors in a number of structural series were actually purified and characterized structurally (Chen et al., (1998), J. C. S. Chem. Commun., 2769-2770; Deng et al, (1999), J. Nat. Prod. 62, 477-480; Deng et al (1999) J. Chem. Soc., Perkin Trans. 1, 1147-1149; Sun et al, (1999) J. Am. Chem. Soc. 121, 6120-6124; Deng et al (1999) J. Nat. Prod. 62, 1000-1002; Sun et al, (1999) J. Nat. Prod. 62, 1110-1113; Deng et al, (1999), J. Nat. Prod. 62, 1624-1626; Ma et al, (1999) J. Nat. Prod. 62, 1660-1663; Deng et al, (2000) BioOrg. Med. Chem. 8, 247-250; Deng et al (2000) J. Nat. Prod. 63, 1356-1360). As a consequence of this more extensive screening, numerous extracts containing putative inhibitors of polymerase β were identified. This included 394 methyl ketone extracts and 247 hexane extracts. While many of these extracts were only weakly active, and some lost inhibitory potential following removal of polyphenols on a polyamide 6S column, hundreds of these extracts, whose identities are known to applicants, have at least some activity as inhibitors of polymerase β. Given the likelihood that polymerases β and σ are similar in overall structure, it is logical to think that they will be subject to inhibition by the same types of compounds.

In fact, this was the experience in screening the polymerase β inhibitors ursolic acid, betulinic acid and 3β-hydroxy-7,24-euphandien-26-oic acid and three additional known polymerase β inhibitors as polymerase σ inhibitors (See FIG. 3). Three of the 6 polymerase β inhibitors were also polymerase σ inhibitors. Critically, the rank orders of potencies against the two enzymes were different and one compound originally identified as a polymerase β inhibitor was actually much (perhaps 50-fold) more potent as an inhibitor of polymerase σ. Accordingly, all of the extracts previously found to exhibit activity against polymerase β will be screened for potential polymerase σ inhibitory activity. Several hundred extracts not found to have polymerase β inhibitory activity will also be screened.

Positive compounds will be immediately put through secondary tests. First, the relative inhibition of pol sigma and pol β will be determined in an highly quantitative and rapid in vitro assay based on filter binding of DNA chains. Further discrimination of potential compounds will be made by determining the IC₅₀ value for pol sigma. The yeast experiments provide proof of principle that nanomolar level inhibitors can be obtained. Compounds with lesser affinity will not be seen as optimal inhibitors. Only compounds that exhibit selective and potent inhibition of polymerase σ will be selected for further study.

Those extracts found to be strongly inhibitory to DNA polymerase σ, but not strongly to polymerase β or other polymerases will be subjected to bioassay-guided fractionation. Of particular interest will be the ability of isolated inhibitors to block the action of polymerase a selectivity, and the ability of these species to potentiate the cytotoxic action of CPT toward cultured cancer cell lines. Fractionation of the extracts will be guided by the same bioassay used to detect polymerase σ activity. This approach was used with great success for the identification of inhibitors of polymerase β. The structure elucidation of the isolated principles will be carried out by spectroscopic methods. These include mass spectrometry, ¹H and ¹³C NMR spectroscopy, as well as 2D NMR spectroscopic methods that permit the molecular framework and stereochemistry of small molecules to be identified.

The same criteria of potency and selectivity will be applied to synthetic compounds identified in the screening of the 2,400 compounds present in the initial National Cancer Institute diversity set. Only those hits exhibiting potency and selectivity will be pursued.

Alternative assays include UV absorbance at 260 nanometers (DNA) in the focused, precipitate in the Z-plane or radioactive nucleotide incorporation (more laborious). Some background from nucleotides may be apparent if focusing is not optimized in the UV assay.

### EXAMPLE 4

Evaluation of Isolated Inhibitors in Mammalian Cells.

The naturally occurring and synthetic inhibitors of human polymerase σ will be evaluated for their ability to potentiate the cytotoxic effects of camptothecin. This will be done in cell culture, similar to the strategy employed to demonstrated potentiation of cisplatin and bleomycin cytotoxicity by polymerase β inhibitors. Specifically, each polymerase σ inhibitor will be used to generate a dose-response curve against P388D₁. Each inhibitor will then be tested at its highest minimally toxic concentration with the highest minimally toxic concentration of camptothecin. Potentiation of the cytotoxic response will be measured via reduction in numbers of viable cells. At least two cell lines will be used to evaluate each compound.

Yeast cell culture assays for pol sigma inhibitors

It has been reported that yeast cells that are partially deficient in pol sigma (trf4 single mutants) are completely dependent on the second pol sigma gene, TRF5, for viability (Castaño et al., 1996B; Castaño et al., 1996A). Thus, compounds that inhibit pol sigma function when the cells express only one form isoform will be preferentially killed over strains that express two isofolms.

This situation can be exploit as follows. Parallel cultures of yeast expressing either: 1) a single human pol sigma-1; or 2) human pol sigma-1 and a second pol sigma gene will be grown in 96 well plates in the presence of libraries of compounds (or later with combinatorial libraries). The human genes are highly likely to function in yeast due to the high conservation and the fact that the protein is functional as a monomeric polypeptide. Human pol sigma-1 and pol sigma-2 have been cloned into yeast expression vectors. It is anticipated that the human genes will fully substitute for the yeast pol sigma gene since, for example, the human topoisomerase I gene is fully functional in yeast, functions as a monomer and shows the same level of evolutionary conservation as pol sigma.

The parallel cultures will then be assayed in the 96-well format for optical density following a predetermined growth period. Compounds that preferentially inhibit the growth of yeast expressing one human pol sigma and that fail to inhibit strains bearing two copies of pol sigma will be taken as positives. Proof of principle is provided by the fact that the topoisomerase poisons would be isolated as positives by this method using cultures expressing one or two yeast isoforms of pol sigma (Walowsky et al., 1999).

In addition, erg6 mutant derivatives of the appropriate yeast strains have been constructed which dramatically increase the permeability of yeast cells to many hydrophobic compounds due to enfeeblement of ergosterol synthesis and the consequent alteration of the cell membranes. To further enhance membrane permeability, yeast cells can be grown in up to 5% DMSO with no adverse effects on growth.

1. A composition comprising an inhibitor of DNA polymerase sigma, wherein said inhibitor has an IC₅₀ in the nanomolar range or lower.

2. The composition of claim 1 further comprising a topoisomerase I inhibitor.

3. The composition of claim 2 wherein the topoisomerase I inhibitor is selected from the group consisting of camptothecin, hycamtin, camptostar, topotecan, 9-amino-camptothecin, 9-amino-10,11 -methylenedioxy-camptothecan and 10,11-methylenedioxy-camptothecan, 7-ethyl- 10-hydroxy 20(S)-camptothecin.

4. A method for potentiating the cytotoxicity of a topoisomerase I inhibitor, said method comprising the step of coadministering a polymerase sigma inhibitor with the topoisomerase I inhibitor.

5. The method of claim 4 wherein the the topoisomerase inhibitor is camptothecin or a camptothecin derivative and the polymerase sigma inhibitor is selected from the group consisting of (24E)-3beta-hydroxy-7,24-euphandien-26-oic acid, ursolic acid and betulinic acid.

6. A pharmaceutical composition comprising a topoisomerase I inhibitor, an inhibitor of polymerase sigma and a pharmaceutically acceptable carrier.

7. The composition of claim 6 wherein the polymerase sigma inhibitor is selected from the group consisting of (24E)-3beta-hydroxy-7,24-euphandien-26-oic acid, ursolic acid and betulinic acid.

8. The composition of claim 7 wherein the topoisomerase inhibitor is camptothecin or a camptothecin derivative.

9. The composition of claim 8 wherein the topoisomerase inhibitor is hycamtin, camptostar or topotecan.

10. A method of treating diseases characterized by undesired cell growth or proliferation, said method comprising the steps of
administering a topoisomerase inhibitor selected from the group consisting of camptothecin or a camptothecin derivative; and
administering a polymerase sigma inhibitor selected from the group consisting of (24E)-3beta-hydroxy-7,24-euphandien-26-oic acid, ursolic acid and betulinic acid.

11. The method of claim 10 wherein the topoisomerase inhibitor is selected from the group consisting of camptothecin, topotecan, 9-amino-camptothecin, 9-amino-10,11-methylenedioxy-camptothecan and 10,11-methylenedioxy-camptothecan, and 7-ethyl-10-hydroxy 20(S)-camptothecin.

12. The method of claim 10 wherein the disease to be treated is a neoplastic disease.

13. The method of claim 10 wherein the topoisomerase inhibitor and the polymerase sigma inhibitor are admixed and administered simultaneously in the form of a single composition.

14. A kit comprising a topoisomerase inhibitor and a polymerase sigma inhibitor.

15 The kit of claim 14, wherein the topoisomerase inhibitor is camptothecin or a camptothecin derivative.

16. The kit of claim 14, wherein the topoisomerase inhibitor is hycamtin, camptostar or topotecan.

17. The kit of claim 15 wherein the polymerase sigma inhibitor is selected from the group consisting of (24E)-3beta-hydroxy-7,24-euphandien-26-oic acid, ursolic acid and betulinic acid.

14. A method of identifying polymerase sigma inhibitors said method comprising the steps of
culturing a first yeast strain that only expresses one isoform of pol sigma;
culturing a second yeast strain that expresses two different isoforms of pol sigma;
contacting the first and second yeast strains with a potential inhibitory compound;
measuring the growth of the first and second yeast strains after a predetermined length of time after the first and second yeast strains were first contacted with the potential inhibitory compound; and
identifying inhibitors based on their ability to inhibit the growth of the first yeast strain relative to the second yeast strain.
